(19) United States
(12) Patent Application Publication
(10) Pub. No.: US 2005/0065436 A1
(43) Pub. Date: Mar. 24, 2005

(54) Rapid and non-invasive optical detection of internal bleeding

(76) Inventors: Winston Zonh Ho, Hacienda Heights, CA (US); Fu-Nan Wang, Hacienda Heights, CA (US); Bo Young Suh, Los Angeles, CA (US)

Correspondence Address: Winston Zonh Ho, 14541 Langhill Drive, Hacienda Heights, CA, 91745, US

(21) Appl. No.: 10/668,801

(22) Filed: Sep. 23, 2003

(51) Int. Cl. A61B 6/00

(52) U.S. Cl. 600/431; 424/9.6

(57) ABSTRACT

A rapid and non-invasive optical method and device for diagnosing internal bleeding or hemorrhage in a human body by detecting leaked blood comprising administering a fluorescent compound parenterally; providing a light beam containing a wavelength absorbable by the florescent compound, wherein the light beam is illuminated at and transmitted through a tissue region into the human body; and analyzing fluorescence signal produced from the fluorescent compound in the leakage of blood for diagnosing the presence or absence of internal bleeding. The invention provides an accurate, rapid, easy-to-use, and inexpensive method for diagnosing internal bleeding, particularly in the fields of gynecology, obstetrics, neonatology, surgery bleeding, post-surgery bleeding, emergency medicine, and veterinary medicine for cases suspected of internal hemorrhage.

### FIELD OF THE INVENTION

The present invention is generally related to a non-invasive optical method and device for in-vivo diagnosing internal bleeding or hemorrhage inside human body with administering a fluorescent compound parenterally, e.g., by intravenous or intra-muscular injection. More particularly, the present invention relates to a method and apparatus that use a light beam and fluorescence signal for diagnosing presence or absence of leakage of blood inside human body. A preferred embodiment of the present invention is directed to using an optical probe device comprising at least one optical fiber light guide and a fluorescent detection means for analyzing fluorescence signal generated from leaked blood in, but not limited to, the abdominal cavity through vaginal canal, cervical region, rectum, or anterior (frontal)/posterior (occipital) fontanel, abdominal wall of infant or other relatively thin tissue of human body, for different reasons and sources of hemorrhage.

### BACKROUND OF THE INVENTION

Internal bleeding is the leakage of blood from blood vessels into spaces in the human body, e.g., intra-peritoneal hemorrhage (i.e., ruptured ectopic pregnancy, ruptured ovarian cyst, hemorrhagic corpus luteum cyst, perforated peptic ulcer disease, hepatic rupture, splenic rupture, any kinds of post-operative bleeding, stab wound injury with continuous bleeding, bowel injuries with continuous bleeding, etc.); intra-cerebral hemorrhage (i.e., intra-cranial or inter-ventricular hemorrhage of newborn, brain contusion/head trauma due to accident, sub-arachnoid hemorrhage); intra-abdominal and/or pelvic hemorrhage secondary to car accident; vitreous hemorrhage of eyes. Internal bleeding caused by injuries, such as blunt force, sharp objects (i.e., knife, gun, broken bone fragments), can damage internal organs and blood vessels.

Internal bleeding is often more serious than external bleeding in certain areas. Internal blood loss, like in intra-cranial space, can pool in surrounding tissues and may build up pressure upon vital organs that cause cardiac and respiratory arrests. Often the signs and symptoms of internal bleeding are less obvious than that of external bleeding. The signs and symptoms of hemorrhage may include pale/cool/clammy skin, thirst, dehydration, rapid pulse, shallow breathing, abdominal pain. Those signs and symptoms are related to the loss of blood acutely or chronically: a rapid blood loss may result in sudden death, whereas a slow blood loss may be neglected by the healthcare professionals and contributes to the loss of life of patients.

Because the sequels of internal bleeding can be very serious, an urgent medical attention including early diagnosis and treatment is mandatory. Unfortunately, there is no any definitive method available at present time without having an exploratory surgery. The conventional methods used include ultrasounds, computerized tomography (CT), magnetic resonance image (MRI), and hormonal analyses, while surgical procedures include laparoscopy and laparotomy. Although ultrasound is a radiation-free technique, it does not provide the nature of fluid character in internal cavities of human body, such as pus, ascites, or blood. The CT emits radiation and does not differentiate blood from other fluids. It is also an expensive procedure. The MRI though, is a radiation-free technique; however, it has similar disadvantages. This method is used to detect soft tissue irregularities. Hormonal analysis (serum beta-hCG and progesterone quantification) is a time consuming assay. The result is not available immediately, especially after regular hours. The invasive procedures such as laparoscopy and laparotomy involve the risks of anesthesia and unnecessary surgery along with complications.

Therefore, there is an urgent need for a method which is accurate, time-saving, rapid, easy-to-use and inexpensive to diagnose internal bleeding, particularly in the fields of gynecology, obstetrics, neonatology (immature and full-term newborn's intra-cranial hemorrhage by examining the anterior or frontal/posterior or occipital fontanel), surgery bleeding, post-surgery bleeding, emergency medicine, and veterinary medicine for cases suspected of internal hemorrhage.

The inability of common diagnostic methodologies for diagnosing internal bleeding has led to developing new methods to detect, localize, and characterize patients with internal bleeding. Fluorescence techniques have been widely used for the analysis of biological samples in clinical assay and biomedical research because of their sensitivity, rapidity and ease of use. However, direct fluorescence measurements in visible and infrared spectral region in whole blood have been almost impossible because of the strong background absorption, scattering and significant autofluorescence. Two highly absorptive components in whole blood are hemoglobin and water. The hemoglobin and water have very strong absorptions at a wavelength of 500-600 nm and 950-1300 nm, respectively. These components significantly reduce the optical penetration depth in addition to the tissue scattering. On the contrary, near infrared (NIR) light, in particularly 600-950 nm, can penetrate tissues much deeper, and blood/tissue autofluorescence and absorption are minimal. Administering a fluorescent solution, mainly, parenterally, e.g., by intravenous injection, the fluorescence compound is quickly transported throughout the body and contained in the bloods vessels. The fluorescent compound can be circulated and distributed to any part of the body within 3-5 minutes. When the fluorescence compound blood is leaked out of blood vessels, it forms a pool of leakage of blood mixture. Fluorescent compound thus provides a marker for detecting leaked blood. By exploring NIR window (600-950 nm) and selected fluorescence compounds, it is possible to detect fluorescence on leakage of blood non-invasively.

The use of NIR window has become increasingly popular in biomedical research. The criteria for non-invasive fluorescence detection from leakage of blood inside human body are as follows: 1. The excitation light beam should be able to penetrate tissues to reach leaked blood; 2. The fluorescent compounds or fluorophores must be able to be excited by an NIR wavelength; and 3. The fluorescence wavelength needs to be in the NIR window, so the fluorescence signal can be detected externally. There are many NIR fluorescent compounds or dyes commercially available. These fluorescence compounds not only absorb NIR light, but also produce fluorescence in NIR window. Examples of NIR dyes are rhodamines, allophycocyanin, phthalocyanines, protoporphyrins, albumin blue, and indocyanine green. Rhodamine dye is used as a laser medium, due to its high fluorescence quantum yield. Phtholocyanines and protoporphyrins are the major components of photodynamic drugs for cancer therapy; these dyes are highly photoactive.

One of the fluorescence compounds, Indocyanine green (ICG), has been used in many clinical applications. Indocyanine green angiography is a diagnostic test, which uses special cameras to photograph the structures in the back of the eye. These tests are very useful for finding leakage or damage to the blood vessels, which nourish the retina (light sensitive tissue). In the test, a colored dye is injected into a vein in the arm of the patient. The dye travels through the circulatory system and reaches the vessels in the retina and those of a deeper tissue layer called the choroid. Indocyanine green fluoresces with invisible infrared light; it requires a special digital camera sensitive to these light rays. Indocyanine green angiography has only recently become a practical technique as these cameras have just become available. Indocyanine green is used as a diagnostic aid for blood volume determination, cardiac output, or hepatic function. After its introduction by Fox et al. (1957) indocyanine green soon came into general use for recording dye dilution curves, in particular for the determination of cardiac output.

U.S. Pat. No. 4,889,129 to Dougherty et al., entire contents of which are incorporated herein by reference, discloses a tumor treatment method to provide and receive radiation from a photodynamic drug in neoplastic tissue. A laser system transmits radiation through an interface into a radiation delivery system, which is in juxtaposition with neoplastic tissue containing a photodynamic drug. The laser system may be a single argon laser pumping a dye laser, two parallel sets of argon lasers pumping a dye laser, a krypton laser or a xenon laser. The interface channels light to radiation sensing devices which are either from a beam splitter indicating the magnitude of the radiation delivered from the laser system to the radiation delivery system or radiation leaking through the light conductor. Luminescent light from the photodynamic drug is selected and provides an indication of drug density and in some cases, depth of the activity.

U.S. Pat. No. 6,180,087 to Achilefu et al., entire contents of which are incorporated herein by reference, discloses an invention relates to compositions of various cyanine and indocyanine dyes wherein novel carbocyclic and heterocyclic moieties are incorporated into the polyene portion of the dye molecules. The sensitivity and specificity of the optical modality can be enhanced by the use of highly absorbing dyes as contrast agents. Particularly, the molecules of the invention are useful for optical diagnostic imaging and therapy, in endoscopic applications for the detection of tumors and other abnormalities, for localized therapy, for photoacoustic tumor imaging, detection and therapy, and for sonofluorescence tumor imaging, detection and therapy.

U.S. Pat. No. 5,196,709 to Berndt et al., entire contents of which are incorporated herein by reference, discloses an invention relating generally to the field of fluorometry and, more particularly, to a method and apparatus for using a laser diode as a source of excitation light for a fluorophore and detecting changes in phase angle and/or modulation of the emitted fluorescence as parameters which correspond to fluorescence lifetimes. A method and apparatus for detecting the change in phase angle and/or modulation of emitted fluorescence of a fluorophore excited by modulated light from a laser diode. The light is both monochromatic and coherent, and can contain harmonic frequency components. The invention provides an inexpensive light excitation source that is small in size, easily manageable, allows for short measurement times, and has lower power requirements.

Although many prior art patents are related to an NIR light source or fluorescence detection, none of them discloses a non-invasive optical method for in vivo diagnosing internal bleeding in human body with administering a fluorescent compound parenterally, e.g., by intravenous injection. More particularly, a preferred embodiment of the present invention is directed to using an optical probe device comprising optical fiber light guide and fluorescent detection means for analyzing fluorescence signal in the leaked blood through vaginal canal, cervical tissue region or rectum to diagnose internal bleeding in human abdomen, or through thin abdominal wall of a baby to detect intra-abdominal bleeding of the baby, or through frontal fontanel/occipital fontanel to investigate intra-cranial bleeding of a newborn, particularly for a premature baby who has the higher incidence of the brain hemorrhage than that of a mature baby, and so on.

### SUMMARY OF THE INVENTION

Accordantly, a non-invasive optical method and device for diagnosing internal bleeding by detecting leakage of blood inside human body comprising administering a fluorescent compound; providing a light source having a light beam, wherein the light beam containing a wavelength absorbed by the fluorescent compound, wherein the light beam is illuminated at and transmitted through a tissue region into the human body; and detecting a fluorescent signal generated from the fluorescent compound in leaked blood for diagnosing the presence or absence of internal bleeding.

Another object of this invention is to provide a non-invasive optical method for diagnosing internal bleeding by detecting leaked blood inside human body comprising administering a fluorescent compound; providing a light source containing a wavelength absorbed by the fluorescent compound, wherein the light source has a wavelength between 600-900 nm; and the fluorescent compound fluoresces a wavelength between 600-900 nm.

Another object of this invention is to provide a non-invasive optical method for diagnosing internal bleeding by detecting leaked blood inside human abdomen and the likes comprising administering a fluorescent compound; providing a light beam containing a wavelength absorbable by the fluorescent compound, wherein the light beam is illuminated at and transmitted through a cervix tissue region/posterior fornix and the likes into the abdomen or other human cavities; detecting a fluorescence signal produced from the fluorescent compound in the leaked blood for diagnosing the presence and absence of internal bleeding in human abdomen or other human cavities.

Another object of this invention is to provide a non-invasive optical device for diagnosing internal bleeding by detecting leaked blood in human body comprising an optical light guide or endoscope containing a light beam with a wavelength absorbable by a fluorescent compound, wherein the fluorescent signal is either an image or a spectral signal; and a fluorescence detecting means comprises at least one optical filter or optical grating, and a detector.

The present non-invasive optics-based probe and medical device has the advantages of simple, real time, and easy operation. The internal bleeding diagnostic device provides rapid and accurate results to assist clinician's decision-making. It should be understood, however, that the detail description and specific examples, while indicating preferred embodiments of the present invention, are given by way of illustration and not of limitation. Further, as will become apparent to those skilled in the art, the teaching of the present invention can be applied to medical devices for measuring fluorescence at a variety of body parts.

### BRIEF DESCRIPTION OF THE DRAWINGS

Additional objects and features of the present invention will become more apparent and the invention itself will be best understood from the following Detailed Description of Exemplary Embodiments, when read with reference to the accompanying drawings.

FIG. 1 is a perspective view of a non-invasive optical probe for in-vivo internal bleeding diagnosis. The fluorescence measurement is based on (a) NIR excitation and (b) fluorescence detection.

FIG. 2 shows an absorption spectrum between 300-1000 nm of an NIR fluorescent compound, Indocyanine green.

FIG. 3 shows fluorescence peaks at 810 nm and spectra between 400-1000 nm in various concentration between 0.5-500 μg/ml of an NIR fluorescent compound, Indocyanine green.

FIG. 4A illustrates a schematic view of non-invasive optical device for internal bleeding diagnostics based on fluorescence spectrum detection with a light guide and optoelectronic system constructed in accordance with the principles of the present invention.

FIG. 4B illustrates a sectional view of an optical fiber-based probe tip according to one of the preferred embodiment.

FIG. 5 illustrates a schematic view of non-invasive optical device for internal bleeding diagnostics based on fluorescence image detection with a light guide, optoelectronic, and endoscopic system constructed in accordance with the principles of the present invention.

### DETAILED DESCRIPTION OF EXEMPLARY EMBODIMENTS

The preferred embodiments of the present invention described below relate particularly to a non-invasive optical method and device for diagnosing internal bleeding or hemorrhage in a human body by detecting leaked blood comprising: administering a fluorescent compound parenterally; providing a light source having a light beam, wherein said light beam contains a wavelength absorbable by said fluorescent compound, wherein said light beam is illuminated at and transmitted through a tissue region into said human body; and after administering said fluorescent compound for a few minutes, analyzing a fluorescence signal produced from said fluorescent compound in said leaked blood for diagnosing the presence or absence of internal bleeding in said human body. While the description sets forth various embodiment specific details, it will be appreciated that the description is illustrative only and should not be construed in any way as limiting the invention. Furthermore, various applications of the invention, and modifications thereto, which may occur to those who are skilled in the art, are also encompassed by the general concepts described below.

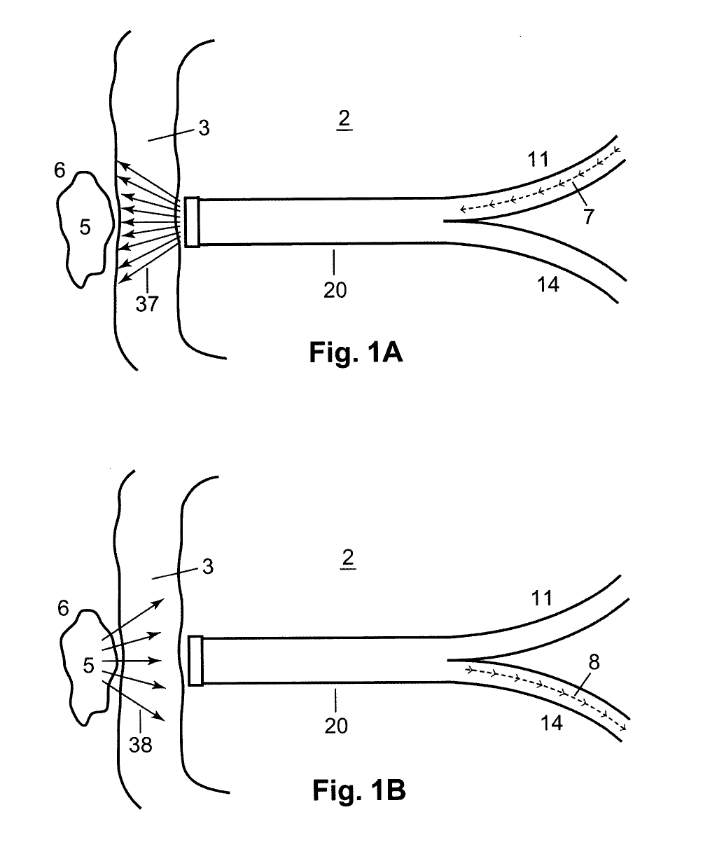

Once a clinician or doctor determines that a patient may have internal bleeding, patients will be administered with fluorescent compound parenterally either intravenously, or intramuscularly (if intravenous injection is not accessible or the case of illness is chronic). The dosage of the fluorescent compound should be effective for producing the fluorescence signal. The typical dosage is in the range of 0.1-10 mg/Kg body weight. Following intravenous or other parenteral administration, the fluorescent compound is quickly transported throughout the body and contained in the bloods vessels. The fluorescent compound can be circulated and distributed to any part of the body within about 3-5 minutes or in a short period of time. If internal bleeding occurs, the blood leaks out the circulation system, as shown in FIG. 1, and proliferates into nearby body cavity 6, such as abdomen. When the leakage of blood accumulated, it forms a pool 5 or a mass of blood. Fluorescent compound thus provides a marker for detecting leaked blood. Internal bleeding occurs frequently in the fields of gynecology, obstetrics, neonatology, surgery bleeding, post-surgery bleeding, emergency medicine, and veterinary medicine.

The fluorescent compound in leaked blood is probed externally with a light beam 7 confined in an optical probe or a light guide 20. The concentration of the fluorescent compound in the blood is in the range of 1-500 μg/ml. Thin tissue with no or minimal capillary blood vessel is the preferred area for optical probing. The potential areas for optical probing are vaginal canal, posterior fornix of vaginal wall, cervical region, rectum, frontal fontanel, occipital fontanel, and other relatively thin layer of human tissue. When the light guide is placed against the tissue, the light beam is penetrated through the tissue 37 to reach the leaked blood. For example, FIG. 1 shows an optical probe 20 is inserted into a vaginal canal 2 and positioned against a cervical tissue/posterior fornix of vaginal wall 3. Cervical tissue area or posterior fornix of vaginal wall is relatively thin, on the order of 2-4 mm. Therefore, the light beam can easily transmit through the tissue and probe the leaked blood 5 in the body cavity 6, such as the cul-de-sac of abdomen. The configuration of the optical probe can be a stand-alone device, or integrated with conventional ultrasound probe, endoscope, fiberscope, or image scope. One preferred embodiment of the optical probe 20 is constructed as a bifurcated optical fibers. The bifurcated fibers combine two ends of illumination fiber 11 and fluorescence detection fiber 14 into an optical probe. The illumination fiber 11 and fluorescence detection fiber 14 can carry the light beam 7 for illumination and collect fluorescence signal 38 into the detection fiber 8, respectively. The optical fiber-based probe has the flexibility to move around in searching for leaked blood or leakage of blood.

Human tissues are highly scattering and absorptive media for ultraviolet and visible light. It is difficult for ultraviolet and visible light to penetrate the tissue more than 5 mm, while near infrared can easily reach 10 mm or more. The employment of NIR photons provides the opportunity to probe deeper tissue layers, excite the fluorophore more effectively, produce more fluorescent photons, and transmit more fluorescence signal for detection. Therefore, the employment of proper wavelength for optical probing and fluorescent compound are critical for this application. The total fluorescence intensity, F, is proportional to the integration of the total fluorescence over the excitation volume V, and is given by the spatial integral of
F(r,θ)=∫Iᵢₙe^{-klr}ε×Q×C×e^{-k2r}×R(r,θ)dr dθ
Where
Iᵢₙ=light intensity at surface of the tissue
K1, K2=extinction coefficients of tissue at excitation and fluorescence wavelengths, respectively
ε=absorption coefficient of fluorescence compound
Q=fluorescence quantum yield of the fluorescence compound
C=concentration of the fluorescence compound in blood
   R(r,θ) is the point source response function, which is a measure of probability that an emitted fluorescence photon generated at position (r,θ) in the sampling volume, V, that will reach the detector at radial position, r, and at the acceptance angle, θ, of the fluorescence collection light guide. This response function can be treated as a conventional rigid rotation function and is dependent on the tissue's optical properties. By proper selecting of excitation light source, a wavelength between 400 nm and 800 nm, and fluorescent compound, a wavelength between 500 nm and 950 nm, it is possible to diagnose internal bleeding non-invasively.

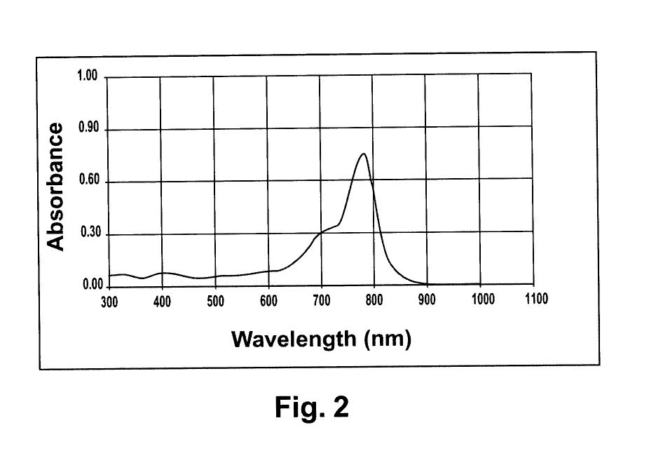

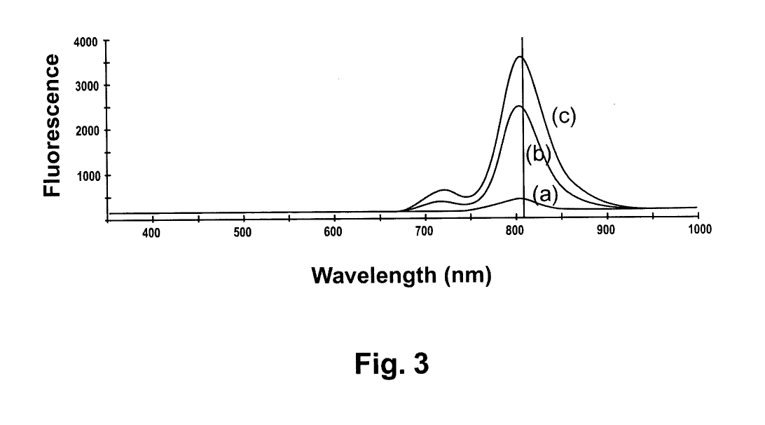

Many NIR fluorescence compounds are potential candidates for the present application. One of examples, indocyanine green (ICG), because of its low toxicity, has been used in many clinical applications. Indocyanine green, molecular weight 775, is a tricarbocyanine type of green dye. FIG. 2 and FIG. 3 show the NIR absorption, 650-850 nm, and NIR fluorescence spectra, 650-900 nm, of ICG, respectively. ICG has little absorption in the visible light. However, it is easily excited by an NIR light source with high quantum efficiency. Diode laser light sources with a wavelength between 630-645 nm is suitable for ICG excitation. The fluorescent peak has a large red shifted relatively to the excitation wavelength. The fluorescence peak at 810 nm is within the NIR window for tissue optics. Due to ICG has a very large fluorescent quantum yield and a distinct peak at 810 nm, a sensitivity of 0.5 μg/ml can be achieved easily. FIG. 3(a), (b), and (c) show the fluorescence spectra of ICG in blood samples with various concentration between 0.5-500 μg/ml

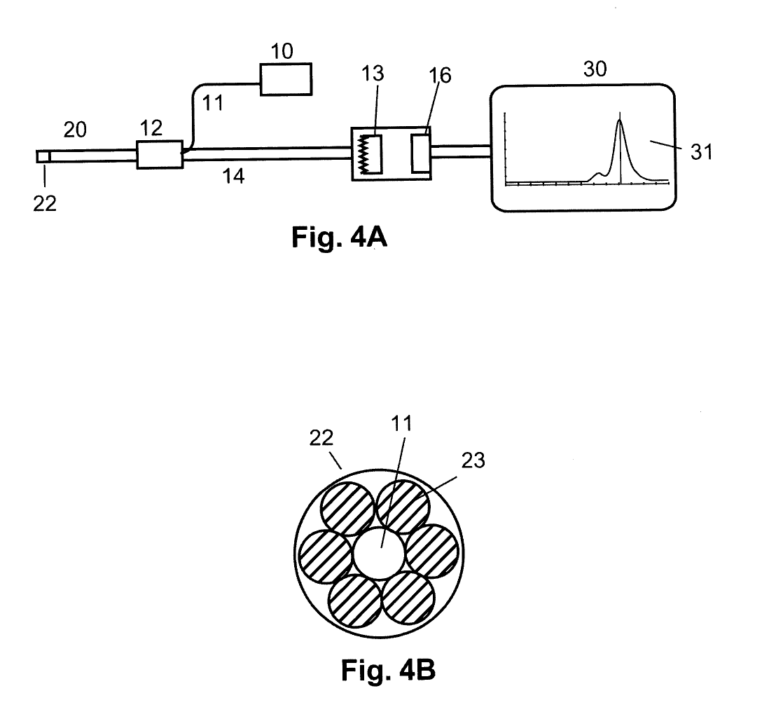

The non-invasive optical probe device for diagnosing internal bleeding, as shown in FIG. 4, is integrated with a light source 10, a fiber splitting coupler 12, an optical probe 20, wavelength diffraction grating 13, a detector 16, and an optical signal analyzing system 30. The light source can be a laser or a lamp. Diode lasers, such as NIR diode lasers with an optical output in the range of 5-50 mw are commercially available. Some lamp sources, which are broadband light sources that cover the entire near infrared range, are also suitable as a continuous light source. Optical band-pass filters or gratings can be used to select a proper narrow band wavelength for excitation. The NIR light beam 7 is coupled into the illumination fiber 11 with a micro lens. Fluorescence signal is collected and delivered to the detection system by the detection fiber 14. The fluorescence signal is either an image or a spectrum. The detection fiber containing a plurality of fibers can improve collection efficiency. The analyzing system 30 displays the fluorescence signature 31 with a distinct fluorescence peak. The spectral signal is physically separated by the diffraction grating 13 and illuminated on a linear CCD 16. Due to the low background in the NIR window, the peak intensity is directly related to the amount of fluorescence compound in leaked blood. The fluorescence peak intensity on CCD is processed by a microprocessor, thus can be correlated to the amount of the leaked blood. FIG. 4B shows one embodiment of the optical probe tip 22; the center fiber is the illumination fiber 11 and the surrounding fibers 23 are fluorescence collection fibers, which form the detection fibers 14.

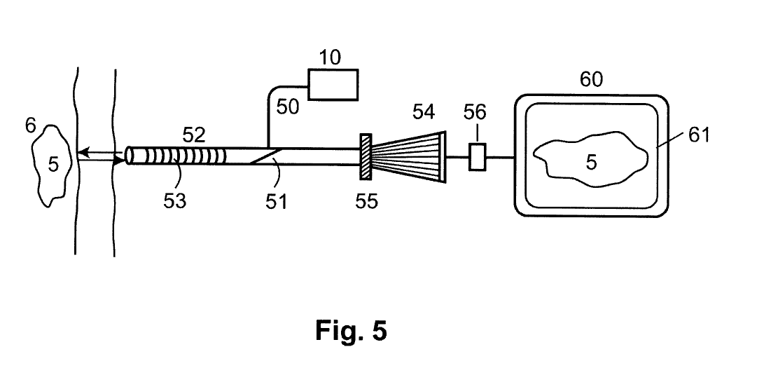

In another preferred embodiment, the light source 10 can be integrated with a conventional endoscope 52 for image detection. As shown in FIG. 5, an NIR light source is coupled into an endoscope, such as a laparoscope, through an optical fiber 50. A 45° mirror 51 reflects the light into the endoscope's lens assembly 53. The fluorescence signal is collected by the endoscope and delivered into a CCD image detector or an image camera 54. An optical filter 55 is installed in front of the NIR sensitive camera. The NIR camera 54 is interfaced through an analog-to-digital converter 56 to an advanced signal processor in a computer 60. The leaked blood 5 in human body is displayed as a pool of leaked blood image 61 on a screen. The real-time data acquisition software supports digital processing with signal normalization. In general, the data acquisition and analysis of the optical parameters are well known to an ordinary person who is skilled in the art.

From the foregoing, it should now be appreciated that an optical probe or light guide containing an illuminating light beam with a wavelength absorbable by a fluorescent compound, wherein the illuminating light beam is transmitted through a tissue region into human body; and a fluorescence detecting means for analyzing a fluorescent signal obtained from the fluorescent compound in blood and for diagnosing the location of internal bleeding in human body, wherein the fluorescence detecting means comprises optical filters or optical gratings or image apparatus. It is also generally applicable for monitoring internal bleeding in many parts of the body. While the invention has been described with reference to a specific embodiment, the description is illustrative of the invention and is not to be construed as limiting the invention. Various modifications and applications may occur to those skilled in the art without departing from the true spirit and scope of the invention as described by the appended claims.

1. A non-invasive optical method for diagnosing internal bleeding or hemorrhage in a human body by detecting leaked blood comprising: administering a fluorescent compound parenterally; providing a light source having a light beam, wherein said light beam contains a wavelength absorbable by said fluorescent compound, wherein said light beam is illuminated at and transmitted through a tissue region into said human body; and after administering said fluorescent compound for a few minutes, analyzing a fluorescence signal produced from said fluorescent compound in said leaked blood for diagnosing the presence or absence of internal bleeding in said human body.

2. The method of claim 1, wherein said leaked blood is selected from a group consisting of internal bleeding for gynecology, obstetrics, neonatology, surgery bleeding, post-surgery bleeding, emergency medicine, and veterinary medicine.

3. The method of claim 1, wherein said tissue region in the human body is selected from a group consisting of vaginal canal, posterior fornix of vaginal wall, cervical region, rectum, frontal fontanel, occipital fontanel, and other relatively thin layer of human tissue.

4. The method of claim 1, wherein said light source has a wavelength between 400 m and 800 mm.

5. The method of claim 1, wherein said fluorescence signal has a wavelength between 500 nm and 950 nm.

6. The method of claim 1, wherein said fluorescent compound has a dosage effective for producing the fluorescence signal.

7. The method of claim 6, wherein said dosage is in the range between 0.1 mg/kg and 10 mg/kg.

8. The method of claim 1, wherein said light source is a laser.

9. The method of claim 1, wherein said fluorescent compound is indocyanine green.

10. The method of claim 1, wherein said fluorescence signal is either an image or a spectral signal.

11. A non-invasive optical device for diagnosing internal bleeding in human body by detecting leaked blood comprising: a fluorescent compound administered parenterally, but not limited to intravenous injection; a light source having a light beam, wherein said light beam contains a wavelength absorbable by said fluorescent compound, wherein said light beam is illuminated at and transmitted through a tissue region into said human body; and fluorescence detection means for analyzing a fluorescence signal produced from said fluorescent compound in said leaked blood for diagnosing the presence or absence of internal bleeding in said human body.

12. The device of claim 11, wherein said leaked blood is selected from a group consisting of internal bleeding for, but not limited to, gynecology, obstetrics, neonatology, surgery bleeding, post-surgery bleeding, emergency medicine, and veterinary medicine.

13. The device of claim 11, wherein said tissue region in the human body is selected from a group consisting of vaginal canal, posterior fornix of vaginal wall, cervical region, rectum, frontal fontanel, occipital fontanel, and other relatively thin layer of human tissue.

14. The device of claim 11, wherein said light source has a wavelength between 400 nm and 800 nm.

15. The device of claim 11, wherein said fluorescence signal has a wavelength between 500 nm and 950 nm.

16. The device of claim 11, wherein said fluorescent compound has a dosage effective for producing the fluorescence signal detectable by the fluorescence detection means.

17. The device of claim 16, wherein said dosage is in the range between 0.1 mg/kg and 10 mg/kg.

18. The device of claim 11, wherein said light source is a laser.

19. The device of claim 11, wherein said fluorescent compound is indocyanine green.

20. The device of claim 11, wherein said fluorescence signal is either an image or a spectral signal.

21. The device of claim 11, wherein said light beam is guided with at least one optical fiber.

22. The device of claim 11, wherein said fluorescence detection means comprises at least one optical filter or optical grating.
